(19) United States
(12) Patent Application Publication
(10) Pub. No.: US 2020/0135340 A1
(43) Pub. Date: Apr. 30, 2020

(54) SYSTEM AND METHOD FOR PREDICTIVE RISK ASSESSMENT AND INTERVENTION

(71) Applicant: Morgan State University, Baltimore, MD (US)

(72) Inventors: Lorece Edwards, Windsor Mill, MD (US); Lawrence Brown, Baltimore, MD (US); Sabriya Dennis, Abingdon, MD (US); Ian Lindong, Baltimore, MD (US)

(21) Appl. No.: 16/668,860

(22) Filed: Oct. 30, 2019

Related U.S. Application Data
(60) Provisional application No. 62/752,530, filed on Oct. 30, 2018.

(51) Int. Cl. G16H 50/30; G16H 10/20; G16H 10/60; G16H 20/00; G06N 5/02

(52) U.S. Cl. CPC G16H 50/30; G16H 10/20; G06N 5/02; G16H 20/00; G16H 10/60

(57) ABSTRACT

Disclosed is a system and method for predictive risk assessment and intervention including a risk assessment unit that receives survey data from a remotely connected survey device. The survey data comprises information about the social and cultural environment of one or more members of a risk population, including the member's perceptions of their social and environmental factors, the member's demographic data, and optionally publicly available data associated with the member's geographic environment. A predictive risk assessment unit analyzes perceived risk hierarchy inventories to generate a risk portfolio for each surveyed member of the population, which risk portfolio may include a risk predictive quotient profile for each such member assigning a numeric value indicating a likelihood of that member engaging in certain negative activities, a recommendation of interventions that are determined to reduce the likelihood of such member engaging in those negative activities, and preferably a record of success and/or failure of various interventions in reducing that risk. Intervention partners then administer the interventions to the surveyed members, record the success or failure of such intervention in preventing the identified dangerous behavior, and transmit an intervention effectiveness report to the predictive risk assessment unit. The predictive risk assessment unit may then modify and recalibrate the survey instrument and the associated recommended intervention products and tools to maximize the successes of interventions.

### CROSS REFERENCE TO RELATED APPLICATIONS

This application is based upon and claims the benefit of U.S. Provisional Application No. 62/752,530 titled “Perceived Risk Hierarchy Methodology,” filed with the United States Patent & Trademark Office on Oct. 30, 2018, the specification of which is incorporated herein by reference in its entirety.

### GOVERNMENT RIGHTS STATEMENT

This invention was made with Government support under contract number SP020188-01 awarded by the United States Substance Abuse and Mental Health Services Administration (SAMHSA). The Government may have certain rights in the invention.

### FIELD OF THE INVENTION

This invention is directed to systems and methods for determining a quantification of an individual's risk of engaging in harmful behaviors and automatically determining mitigating interventions to reduce such risk, and more particularly to computer-implemented systems and methods for creating electronic individual risk portfolios based on an individual's perceived risks and their cultural and social environment and analyzing such electronic individual risk portfolios to automatically determine and distribute interventions to intervention partners for mitigating the risk of engaging in such harmful behaviors.

### BACKGROUND OF THE INVENTION

A number of populations throughout the United States, and in fact throughout the world, face various dangers that are brought about by their own behaviors and activities. The likelihood of their participation in those danger-prone behaviors and activities has been found to relate at least partially to their perception of various risks that they face in their daily lives. Further, those perceptions of the risks that they face in their lives are shaped by their own cultural and social environments and interactions.

For example, it has been found that when approaching emerging adults with respect to HIV-risk behaviors and perceptions, even with the best of incentives, emerging adults in certain urban populations were not testing for HIV and did not perceive communicable disease as a concern, and did not consider themselves at risk of contracting a communicable disease. It has been found that an individual's perception of these and other risks shapes that individuals' activities, and in fact perceptions about certain risks may tend to induce high-risk behaviors in certain individuals. For instance, it has been found that youth and emerging adults' perception of health risk or severity is attenuated by what they perceive as more imminent and immediate risks, such as matters of personal safety, danger, and perceived survival expectations. As a result, the individual's perceived risk hierarchy can influence the individual's likelihood of engaging in negative behaviors, such as: (1) indifference to sexually transmitted infections (STI), HIV and HCV prevention screening that have been made significantly more convenient and accessible compared to traditional testing and screening modalities; (2) a low perception of STI, HIV and HCV risk despite engagement in high-risk behaviors, and (3) knowingly engaging in these behaviors despite knowledge of perils and potential detrimental outcomes. Additionally, it has been found that the individual's survival expectations are linked to a range of authentic concerns (e.g., internalized symptoms such as depression, anxiety, fear, and hopelessness) and externalizing behaviors, such as aggression. The inventors herein have found that certain populations live each day on high alert for perceived threats and are in a constant state of mobilizing for fight, flight, or suspense as they anticipate the next assault, such that it becomes less likely for them to concentrate, learn, recall, do well in school, consider employment, perceive a future orientation, and delay immediate gratification. As a result, they are more likely to absorb and trivialize health and other risks that are otherwise real and can have deleterious impact. However, intervention by health and other professionals may likewise modify the individual's perceptions regarding risk, and in turn modify their behavior to move away from high risk behaviors. Nonetheless, given the difficulty in reaching large numbers of at-risk populations, there is only limited success in achieving behavior modification in this way.

As an individual's perceived risk hierarchy may impact the likelihood that they will engage in certain dangerous behaviors, and as intervention tools do exist (such as counseling, education, etc.) that can help to adjust the individual's perceptions of the risks they face in daily life, it would be advantageous to provide systems and methods by which data may be collected from larger portions of at-risk populations to evaluate their perceived risk hierarchy, and by which intervention tools may be automatically suggested for use by intervention service providers (e.g., counselors, medical professionals, etc.) and their successes tracked in order to reduce the likelihood that such individuals will engage in the negative activities.

### SUMMARY OF THE INVENTION

Disclosed herein is a system and method for predictive risk assessment and intervention that avoids one or more disadvantages of the prior art. A system is described herein having a computer-implemented predictive risk assessment unit that receives survey data from a remotely connected survey device. The survey data comprises information about the social and cultural environment of one or more members of a risk population to create a digital perceived risk hierarchy inventory for each surveyed population member, which may include data such as the member's perceptions of their social and environmental factors (e.g., police contact, community violence, experience with substance abuse, exposure to sexually transmitted diseases, etc.), the member's demographic data (e.g., age, gender, education level, etc.), and publicly available data associated with the member's geographic environment (e.g., local homelessness, drug treatment, arrests, etc.). The digital perceived risk hierarchy is transmitted from the remote survey device to the predictive risk assessment unit, where a risk determination engine analyzes each such perceived risk hierarchy inventory to generate a risk portfolio for each surveyed member of the population, which risk portfolio may include a risk predictive quotient profile for each such member assigning a numeric value indicating a likelihood of that member engaging in certain negative activities, a recommendation of interventions that are determined to reduce the likelihood of such member engaging in those negative activities, and preferably a record of success and/or failure of various interventions in reducing that risk. At least a portion of the risk portfolio (including at least the recommendation of interventions) is then transmitted from the predictive risk assessment unit to one or more intervention partners who administer the interventions to the surveyed members, record the success or failure of such intervention in preventing the identified dangerous behavior, and transmit an intervention effectiveness report to the predictive risk assessment unit. The predictive risk assessment unit may then modify and recalibrate the survey instrument (and particularly weights assigned to various elements of the digital perceived risk hierarchy inventory) and the associated recommended intervention products and tools using various analytical methods to maximize the successes of interventions, particularly as evidenced in further intervention effectiveness reports received form intervention partners.

Systems and methods configured in accordance with certain aspects of the invention provide a snapshot of the cultural and social environment of the member of the risk population, and provide an insight into the future possibility of that population member engaging in at-risk behaviors or activities. Thus, such systems and methods may provide strong indications of where intervention planning can be employed to mitigate such behaviors. Likewise, the results of actual interventions may suggest whether refinement of the survey or the analytic methods performed by the predictive risk assessment unit is necessary or warranted.

In accordance with certain aspects of an embodiment of the invention, a computer method is disclosed for predictive risk assessment and intervention, comprising the steps of: providing a predictive risk assessment unit having a processor and a memory; providing a survey device in data communication with the predictive risk assessment unit; receiving at the predictive risk assessment unit from the survey device a digital perceived risk hierarchy inventory associated with a human risk population member; analyzing at the processor of the predictive risk assessment unit the digital perceived risk hierarchy inventory to identify one or more intervention products that are calculated to mitigate one or more negative or harmful behaviors associated with the perceived risk hierarchy inventory; and storing in the memory an individual risk portfolio associated with the human risk population member, the individual risk portfolio including the one or more intervention products.

In accordance with further aspects of an embodiment of the invention, a computer system is disclosed for predictive assessment and intervention, comprising: a predictive risk assessment unit having a processor and a memory; and a survey device in data communication with the predictive risk assessment unit; wherein the processor of the predictive risk assessment unit includes computer instructions configured to: receive from the survey device a digital perceived risk hierarchy inventory associated with a human risk population member; analyze the digital perceived risk hierarchy inventory to identify one or more intervention products that are calculated to mitigate one or more negative or harmful behaviors associated with the perceived risk hierarchy inventory; and store in the memory an individual risk portfolio associated with the human risk population member, the individual risk portfolio including the one or more intervention products.

Still other aspects, features and advantages of the invention are readily apparent from the following detailed description, simply by illustrating a number of particular embodiments and implementations, including the best mode contemplated for carrying out the invention. The invention is also capable of other and different embodiments, and its several details can be modified in various obvious respects, all without departing from the spirit and scope of the invention. Accordingly, the drawings and description are to be regarded as illustrative in nature, and not as restrictive.

### BRIEF DESCRIPTION OF THE DRAWINGS

The numerous advantages of the present invention may be better understood by those skilled in the art by reference to the accompanying drawings in which:

FIG. 1 is a schematic view of a system for predictive risk assessment and intervention in accordance with certain aspects of an embodiment of the invention.

FIG. 2 is a representation of a risk predictive quotient matrix for use with the system of FIG. 1.

FIG. 3 is a schematic flowchart of a method for predictive risk assessment and intervention in accordance with further aspects of an embodiment of the invention.

FIG. 4 is a schematic view of a computing device for use with the system of FIG. 1.

### DETAILED DESCRIPTION

The invention summarized above may be better understood by referring to the following description, claims, and accompanying drawings. This description of an embodiment, set out below to enable one to practice an implementation of the invention, is not intended to limit the preferred embodiment, but to serve as a particular example thereof. Those skilled in the art should appreciate that they may readily use the conception and specific embodiments disclosed as a basis for modifying or designing other methods and systems for carrying out the same purposes of the present invention. Those skilled in the art should also realize that such equivalent assemblies do not depart from the spirit and scope of the invention in its broadest form.

Descriptions of well-known functions and structures are omitted to enhance clarity and conciseness. The terminology used herein is for the purpose of describing particular embodiments only and is not intended to be limiting of the present disclosure. As used herein, the singular forms “a”, “an” and “the” are intended to include the plural forms as well, unless the context clearly indicates otherwise. Furthermore, the use of the terms a, an, etc. does not denote a limitation of quantity, but rather denotes the presence of at least one of the referenced items.

The use of the terms “first”, “second”, and the like does not imply any particular order, but they are included to identify individual elements. Moreover, the use of the terms first, second, etc. does not denote any order of importance, but rather the terms first, second, etc. are used to distinguish one element from another. It will be further understood that the terms “comprises” and/or “comprising”, or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Although some features may be described with respect to individual exemplary embodiments, aspects need not be limited thereto such that features from one or more exemplary embodiments may be combinable with other features from one or more exemplary embodiments.

To the knowledge of the inventors herein, the combination of perception, risk, and hierarchy have not previously been addressed in a manner that may be used to automatically connect factors addressing a risk population member's environment (e.g., an urban environment), the resultant risks that they face based on the cultural and social environment characteristics of that environment, and the interventions that may best mitigate those risks. The systems and methods employed herein are based upon a finding that emerging adults prioritize risk within their own framework for survival and success. The priority risk should be acknowledged, addressed, and satisfied so that the emerging adults can proceed to practice prevention, observe proper practices and behaviors, focus on positive short and long term goals, increase academic performance and attain educational goals, as well as maintain a positive orientation going forward. As disclosed in greater detail below, the perceived risk hierarchy inventory of a surveyed emerging adult member of the risk population is analyzed at a predictive risk assessment unit to develop a behavior risk profile and assessment and, based upon such behavior risk profile and assessment, automatically assign recommended intervention products that are determined to reduce the member's risk of engaging in harmful behavior. Such recommended intervention products (and preferably other elements of the risk population member's behavior risk profile and assessment) are transmitted to at least one intervention partner (e.g., educational institutions, community centers, hospitals, counselors, physician's offices, etc.) to allow that partner to use and track the success of intervention measures, and transmit back to the predictive risk assessment unit an intervention effectiveness report which may be used to further refine the automated analytical tools used to evaluate the member's risk of engaging in harmful behavior.

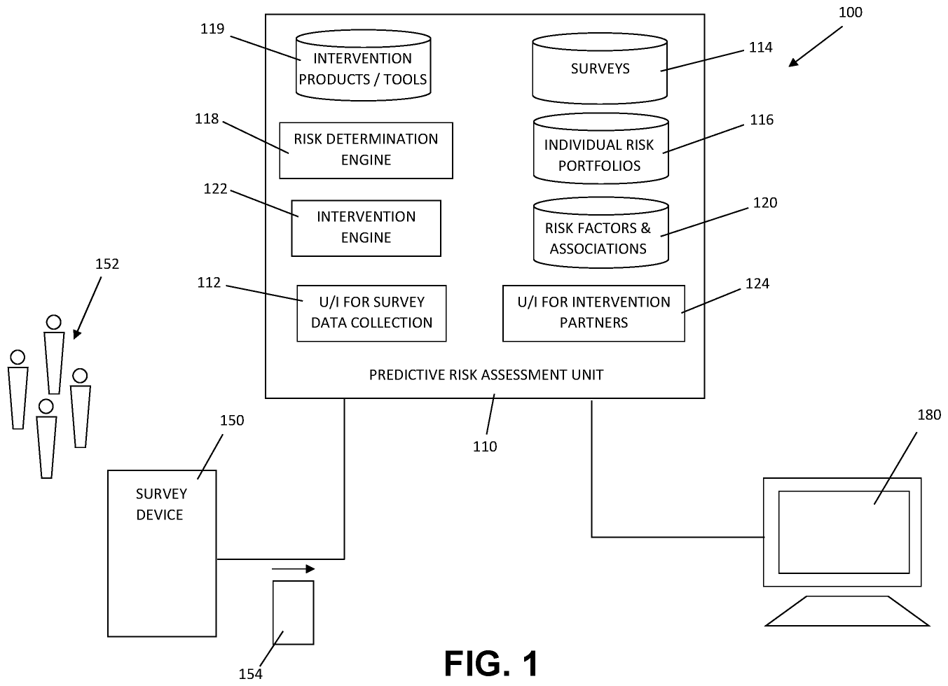

FIG. 1 shows an exemplary schematic representation of a system for predictive risk assessment and intervention (shown generally at 100) including a predictive risk assessment unit 110, one or more computer implemented remote survey devices 150 in remote data communication with predictive risk assessment unit 110, and one or more intervention partner computers 180 in data communication with predictive risk assessment unit 110. Predictive risk assessment unit 110 is preferably a hosted system that may, by way of non-limiting example, be hosted in a cloud processing environment accessible via a wide area data network such as the Internet.

Survey device 150 is preferably a remote computing device, such as a tablet, a laptop computer, a smartphone, or similarly configured readily portable computing device, configured for remote communication with predictive risk assessment unit 110. Survey device 150 is used to record responses from one or more members of risk population 152 at least relating to each such surveyed member's perceived risk hierarchy, and preferably also relating to certain demographic data relating to such surveyed member. Predictive risk assessment unit 110 preferably hosts a user interface 112 for survey data collection, which preferably receives a login request from survey device 150 and authenticates the user (e.g., through password entry or other such authentication methods as may be chosen by those skilled in the art) to predictive risk assessment unit 110. Predictive risk assessment unit 110 may receive a request from survey device 150 for a survey from survey database 114, and may transmit a digital survey to survey device 150 for administering to the member of risk population 152. Preferably, the digital survey includes survey questions that solicit the population member's perception of various cultural and social factors in their day-to-day environment, in addition to certain demographic data relating to that risk population member. The digital survey may thus collect experiential perception profile data, including (by way of non-limiting example) data indicative of the risk population member's perception of and exposure to police contact, negative community experiences (e.g., HIV/AIDS and other sexually transmitted diseases, mental health impairment, substance abuse, community violence, etc.). The digital survey may additionally collect individual demographic profile data, including (by way of non-limiting example) data indicative of the risk population member's employment status (e.g., unemployed, employed part-time, employed full-time), education level (e.g., high school, college, post-graduate education), zip code (or smaller geographic designation), voter registration status, age, gender, sexual orientation, and church attendance. Of course, other experiential perception profile data and demographic profile data may be included in the digital survey as may be preferably for a given risk population, which may be readily determined by persons skilled in the art.

The data collected by the digital survey may form a perceived risk hierarchy inventory 154 that may be transmitted from survey device 150 to predictive risk assessment unit 110 through user interface 112, and risk assessment unit 110 may generate and store in data memory an individual risk portfolio 116 for the surveyed risk population member 152 that includes the member's perceived risk hierarchy inventory 154 (i.e., their experiential perception profile data and demographic profile data). Optionally, predictive risk assessment unit 110 may supplement the member's individual risk portfolio 116 with existing public data that may be geospatial in nature (e.g., publicly available community demographic data), which may be helpful to further determine interrelationships among various cultural and social factors that may affect a risk population member's likelihood of engaging in dangerous or high-risk behaviors.

Using statistical analytical methods as may be selected and customized by those skilled in the art, a risk determination engine 118 may analyze the surveyed member's experiential perception profile data and demographic profile data collected in their individual risk profile 116, and may generate a risk predictive quotient matrix 156 for a variety of risk segments for that risk population member 152. By way of non-limiting example, the risk segments may include sexual health factors (e.g., HIV and other sexually transmitted diseases and teen pregnancy), mental health factors (e.g., substance abuse, suicide and depression), and violence and injury factors (e.g., gun violence, domestic violence and child abuse). Once generated, the risk predictive quotient for that risk population member 152 may then be added to and stored with the member's individual risk portfolio 116.

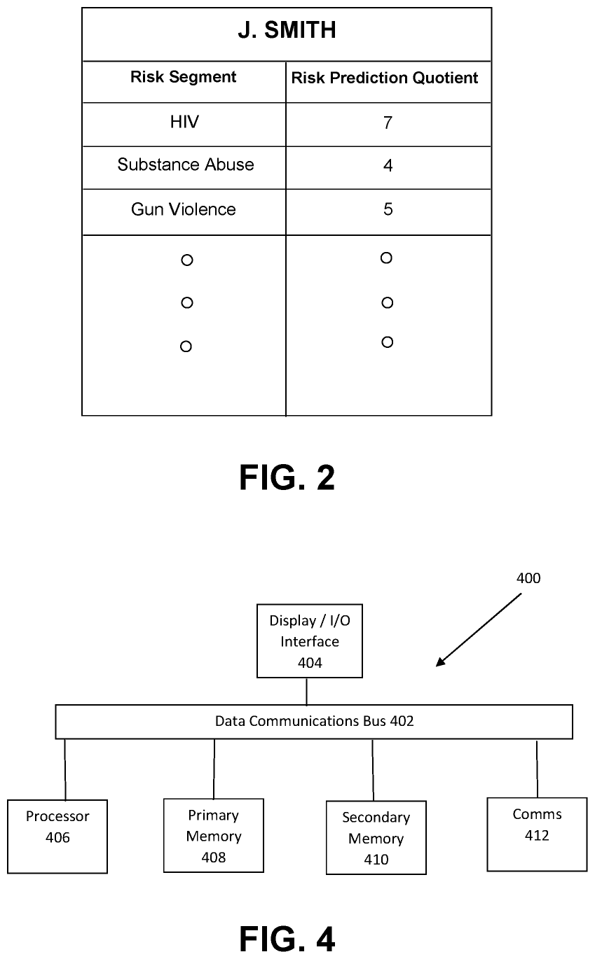

As shown in FIG. 2, the predictive quotient matrix 156 for the risk population member 152 may include multiple risk segments 157 for which user perception data were collected in perceived risk hierarchy inventory 154, and an associated, calculated, numerical risk prediction quotient 158 for each risk segment 157. Varying weights may be applied by risk determination engine 118 to the risk segments 157 in the risk population member's predictive quotient matrix 156 based on discovered interrelationships between perceived risks and actual risks experienced by individuals that are similarly situated to the surveyed member 152 (e.g., of similar race, neighborhood, age, or other demographic characteristics). The weighted values of the risk population member's risk predictive quotient matrix 156 may then be run through a combination or menu of statistical tests and/or a Bayesian or comparable mathematical methods (which statistical methods are well known to those skilled in the art) applied by risk determination engine 118 to single out the greatest areas of risk and those interrelationships that would benefit from intervention, such as through application of intervention products and tools 119. The risk population member's risk portfolio may thus provide a ranking and prioritizing of risk outcomes based on a set of values, beliefs/attitudes and knowledge, and the input data received from perceived risk hierarchy inventory 154 may be used to calculate the level of risk of participation in certain high-risk behaviors. More particularly, the risk determination engine 118 may analyze the risk population member's risk portfolio 116 to generate a value indicative of the risk that such risk population member 152 will engage in the identified risk-associated behavior or activity. Further, where such value for a given risk-associated behavior exceeds a predetermined value that may be selected and adjusted by a system administrator for varying risk populations, predictive risk assessment unit 110 may assign one or more intervention products 119 to the member's risk portfolio 116, which intervention product has been determined to reduce the risk of the member engaging in such risk-associated behavior.

Preferably, a database of risk factors and associations 120 is provided that defines interrelationships among the various risk factors that are analyzed by risk determination engine 118, which interrelationships are preferably expressed in an index that can be displayed in tabular form or graphically, as in (by way of non-limiting example) a Geographical Information System (GIS). Such database of risk factors and associations 120 may be updated through ongoing direct contact with risk population members 152. More particularly, through direct contact with residents of multiple neighborhoods, inter-relationships between variations of perceived risks versus profile data may be used to define and continuously update thresholds that can be displayed in a tabular fashion, in a “heat map,” or in such other visual presentation as may be preferred by those skilled in the art.

With continuing reference to FIG. 1, predictive assessment unit 110 also provides an intervention engine 122 preferably capable of (i) determining an intervention product or tool 119 that has been determined to mitigate the risk of a risk population member 152 engaging in a harmful behavior and assigning such intervention product or tool 119 to the risk population member's individual risk portfolio 116, and (ii) modifying the determination of what intervention product or tool 119 may mitigate a given risk based upon a measured effectiveness of an applied intervention product or tool 119 by an intervention partner 180. More particularly, intervention engine 122 analyzes a risk population member's individual risk portfolio 116 to automatically assign an intervention from intervention products/tools 119, such as (by way of non-limiting example) one or more interventions 119 that have been determined as helpful to minimize risk and prevent future disabling social phenomena and/or other negative incident. The risk population member's risk predictive quotient matrix 156 (FIG. 2) is used by the risk determination engine 118 to determine the level of associated risk based on the risk prediction quotient 158 (e.g., high-risk critical, high-risk non-critical, moderate-risk, low-risk, etc.), and uses that level of associated risk to select and assign intervention products/tools 119 in an effort to reduce that risk. Optionally, data collected from multiple neighborhoods and the interrelationships between various perceived risks versus risk population member risk portfolio (which as mentioned above may be used to generate a heat map or table) may also be used to select and assign intervention products/tools 119 to similarly-situated risk population members 152, which may be particularly helpful where individual members 152 have not been surveyed and/or had an individual risk portfolio 116 established by predictive risk assessment unit 110.

With further reference to FIG. 1, intervention partner computers 180 are also preferably in remote data communication with predictive risk assessment unit 110. Intervention partner computers 180 engage with predictive risk assessment unit 110 through an intervention partner user interface 124, and after authentication (such as by password protected login or such other authentication method as may be selected by those skilled in the art) may select an individual risk portfolio 116 for a member of risk population 152 that the associated intervention partner is serving (e.g., e.g., educational institutions, community centers, hospitals, counselors, physician's offices, etc.). Intervention partner computers 180 may receive one or more intervention products 119 that are assigned to the individual risk portfolio 116 of their respective risk population member 152, such that an intervention partner user of intervention partner computer 180 may oversee the administration of such intervention product/tool 119. Based upon that intervention partner user's observations of risk population member 152 after administration of the associated intervention product/tool 119, the user may transmit from intervention partner computer 180 data reporting the success of administration of the respective intervention product/tool 119 (e.g., a numeric score indicating a designated level of success, which may vary from behavior to behavior) in mitigating the particular dangerous or harmful behavior or activity, and the success score for the applied intervention product/tool 119 may be assigned to the respective risk population member's individual risk portfolio 116.

Based upon the results of the intervention product/tool 119 applied to the respective risk population member 152 (as evidenced by the numeric score assigned to such intervention product/tool 119), and as part of a feedback process, intervention engine 122 may make further adjustments in weightings applied to the risk segments 157 in the risk population member's predictive quotient matrix 156. Questions in the survey applied by survey device 150 may be added or subtracted to change particular weights assigned to various elements of the digital perceived risk hierarchy inventory 154, and different statistical methods or algorithms may be applied to the analysis by intervention engine 122. Moreover, as part of an ongoing data collection effort, predictive risk assessment unit 110 may carry out further iterations to reflect the new line of questions that will be posed to the risk population. Still further, as part of the feedback process, the results of the adjustments in weighting made by intervention engine 122 may be compared to actual results of a given intervention product/tool 119 to validate the risk index/probability assessment.

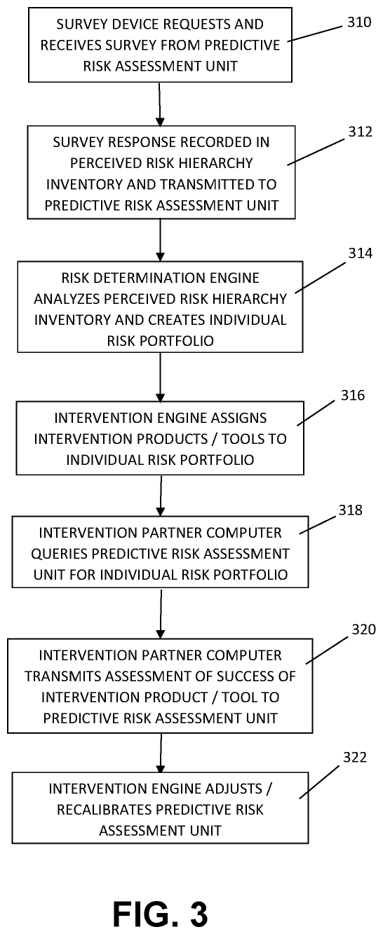

Next, FIG. 3 is a schematic view of a computer-implemented process for predictive risk assessment and intervention in accordance with further aspects of an embodiment. At step 310, an authenticated user of survey device 150 causes survey device 150 to request a survey 114 from predictive risk assessment unit 110, which survey 114 is then transmitted to survey device 150 for administration to a risk population member 152. Next at step 312, the risk population member's responses to survey 114 are recorded in a perceived risk hierarchy inventory 154 which is transmitted from survey device 150 to predictive risk assessment unit 110. At step 314, risk determination engine 118 analyzes the perceived risk hierarchy inventory 154 and establishes an individual risk portfolio 116 for the respective risk population member 152, which includes a risk predictive quotient matrix assigning a numeric risk prediction quotient 158 to each of one or more risk segments 157. At step 316, intervention engine 122 assigns one or more intervention products/tools 119 to the individual risk portfolio 116 for that risk population member, which intervention products/tools 119 are calculated as being able to mitigate the risk of that risk population member 152 engaging in a dangerous behavior or activity. At step 318, an intervention partner computer 180 queries predictive risk assessment unit 110 to obtain the individual risk portfolio 116 for the risk population member 152 that they are servicing, and thereafter may administer the intervention products/tools 119 associated with that member's individual risk portfolio 116. Next, at step 320, the intervention partner computer 180 transmits to predictive risk assessment unit 110 an assessment of the success of application of the intervention product/tool 119 in mitigating the risk of the risk population member engaging in a particularly dangerous or harmful activity. At step 322, intervention engine evaluates the assessment received from intervention partner computer 180, and optionally adjusts and/or recalibrates the weights assigned to differing risk segments in the risk population member's risk predictive quotient matrix, and/or modifies the questions presented by survey device 150 and statistical methods applied by risk determination engine 118 to further refine the risk population member's individual risk portfolio 116.

Those skilled in the art will recognize that each of predictive risk assessment unit 110, survey device 150, and intervention partners 180 may each take the form of computer system 400 as reflected in FIG. 4, though variations thereof may readily be implemented by persons skilled in the art as may be desirable for any particular installation. In each such case, one or more computer systems 400 may carry out the foregoing methods as computer code.

Computer system 400 includes a communications bus 402, or other communications infrastructure, which communicates data to other elements of computer system 400. For example, communications bus 402 may communicate data (e.g., text, graphics, video, other data) between bus 402 and an I/O interface 404, which may include a display, a data entry device such as a keyboard, touch screen, mouse, or the like, and any other peripheral devices capable of entering and/or viewing data as may be apparent to those skilled in the art. Further, computer system 400 includes a processor 406, which may comprise a special purpose or a general purpose digital signal processor. Still further, computer system 400 includes a primary memory 408, which may include by way of non-limiting example random access memory (“RAM”), read-only memory (“ROM”), one or more mass storage devices, or any combination of tangible, non-transitory memory. Still further, computer system 400 includes a secondary memory 410, which may comprise a hard disk, a removable data storage unit, or any combination of tangible, non-transitory memory. Finally, computer system 400 may include a communications interface 412, such as a modem, a network interface (e.g., an Ethernet card or cable), a communications port, a PCMCIA slot and card, a wired or wireless communications system (such as Wi-Fi, Bluetooth, Infrared, and the like), local area networks, wide area networks, intranets, and the like.

Each of primary memory 408, secondary memory 410, communications interface 412, and combinations of the foregoing may function as a computer usable storage medium or computer readable storage medium to store and/or access computer software including computer instructions. For example, computer programs or other instructions may be loaded into the computer system 400 such as through a removable data storage device (e.g., a floppy disk, ZIP disks, magnetic tape, portable flash drive, optical disk such as a CD, DVD, or Blu-ray disk, Micro Electro Mechanical Systems (“MEMS”), and the like). Thus, computer software including computer instructions may be transferred from, e.g., a removable storage or hard disc to secondary memory 410, or through data communication bus 402 to primary memory 408.

Communication interface 412 allows software, instructions and data to be transferred between the computer system 400 and external devices or external networks. Software, instructions, and/or data transferred by the communication interface 412 are typically in the form of signals that may be electronic, electromagnetic, optical or other signals capable of being sent and received by communication interface 412. Signals may be sent and received using a cable or wire, fiber optics, telephone line, cellular telephone connection, radio frequency (“RF”) communication, wireless communication, or other communication channels as will occur to those of ordinary skill in the art.

Computer programs, when executed, allow processor 406 of computer system 400 to implement the methods discussed herein for predictive risk assessment and intervention according to computer software including instructions.

Computer system 400 may perform any one of, or any combination of, the steps of any of the methods described herein. It is also contemplated that the methods according to the present invention may be performed automatically, or may be accomplished by some form of manual intervention.

The computer system 400 of FIG. 3 is provided only for purposes of illustration, such that the invention is not limited to this specific embodiment. Persons having ordinary skill in the art are capable of programming and implementing the instant invention using any computer system.

Further, computer system 400 may, in certain implementations, comprise a handheld device and may include any small-sized computing device, including by way of non-limiting example a cellular telephone, a smartphone or other smart handheld computing device, a personal digital assistant, a laptop or notebook computer, a tablet computer, a hand held console, an MP3 player, or other similarly configured small-size, portable computing device as may occur to those skilled in the art.

As explained above, the system of FIG. 1 may, in an exemplary configuration, be implemented in a cloud computing environment for carrying out the methods described herein. That cloud computing environment uses the resources from various networks as a collective virtual computer, where the services and applications can run independently from a particular computer or server configuration making hardware less important. The cloud computer environment includes at least one survey device 150 operating as a client computer. The client computer may be any device that may be used to access a distributed computing environment to perform the methods disclosed herein, and may include (by way of non-limiting example) a desktop computer, a portable computer, a mobile phone, a personal digital assistant, a tablet computer, or any similarly configured computing device. That client computer preferably includes memory such as RAM, ROM, one or more mass storage devices, or any combination of the foregoing. The memory functions as a computer readable storage medium to store and/or access computer software and/or instructions.

That client computer also preferably includes a communications interface, such as a modem, a network interface (e.g., an Ethernet card), a communications port, a PCMCIA slot and card, wired or wireless systems, and the like. The communications interface allows communication through transferred signals between the client computer and external devices including networks such as the Internet and a cloud data center. Communication may be implemented using wireless or wired capability, including (by way of non-limiting example) cable, fiber optics, telephone line, cellular telephone, radio waves or other communications channels as may occur to those skilled in the art.

Such client computer establishes communication with the one more servers via, for example, the Internet, to in turn establish communication with one or more cloud data centers that implement predictive risk assessment and intervention system 100. A cloud data center may include one or more networks that are managed through a cloud management system. Each such network includes resource servers that permit access to a collection of computing resources and components of predictive risk assessment and intervention system 100, which computing resources and components can be invoked to instantiate a virtual computer, process, or other resource for a limited or defined duration. For example, one group of resource servers can host and serve an operating system or components thereof to deliver and instantiate a virtual computer. Another group of resource servers can accept requests to host computing cycles or processor time, to supply a defined level of processing power for a virtual computer. Another group of resource servers can host and serve applications to load on an instantiation of a virtual computer, such as an email client, a browser application, a messaging application, or other applications or software.

The cloud management system may comprise a dedicated or centralized server and/or other software, hardware, and network tools to communicate with one or more networks, such as the Internet or other public or private network, and their associated sets of resource servers. The cloud management system may be configured to query and identify the computing resources and components managed by the set of resource servers needed and available for use in the cloud data center. More particularly, the cloud management system may be configured to identify the hardware resources and components such as type and amount of processing power, type and amount of memory, type and amount of storage, type and amount of network bandwidth and the like, of the set of resource servers needed and available for use in the cloud data center. The cloud management system can also be configured to identify the software resources and components, such as type of operating system, application programs, etc., of the set of resource servers needed and available for use in the cloud data center.

In accordance with still further aspects of an embodiment of the invention, a computer program product may be provided to provide software to the cloud computing environment. Computer products store software on any computer useable medium, known now or in the future. Such software, when executed, may implement the methods according to certain embodiments of the invention. By way of non-limiting example, such computer usable mediums may include primary storage devices (e.g., any type of random access memory), secondary storage devices (e.g., hard drives, floppy disks, CD ROMS, ZIP disks, tapes, magnetic storage devices, optical storage devices, MEMS, nanotech storage devices, etc.), and communication mediums (e.g., wired and wireless communications networks, local area networks, wide area networks, intranets, etc.). Those skilled in the art will recognize that the embodiments described herein may be implemented using software, hardware, firmware, or combinations thereof.

The cloud computing environment described above is provided only for purposes of illustration and does not limit the invention to this specific embodiment. It will be appreciated that those skilled in the art are readily able to program and implement the invention using any computer system or network architecture.

Having now fully set forth the preferred embodiments and certain modifications of the concept underlying the present invention, various other embodiments as well as certain variations and modifications of the embodiments herein shown and described will obviously occur to those skilled in the art upon becoming familiar with said underlying concept. It should be understood, therefore, that the invention may be practiced otherwise than as specifically set forth herein.

What is claimed is:

1. A computer method for predictive risk assessment and intervention, comprising the steps of:
providing a predictive risk assessment unit having a processor and a memory;
providing a survey device in data communication with said predictive risk assessment unit;
receiving at said predictive risk assessment unit from said survey device a digital perceived risk hierarchy inventory associated with a human risk population member;
analyzing at said processor of said predictive risk assessment unit said digital perceived risk hierarchy inventory to identify one or more intervention products that are calculated to mitigate one or more negative or harmful behaviors associated with said perceived risk hierarchy inventory; and
storing in said memory an individual risk portfolio associated with said human risk population member, said individual risk portfolio including said one or more intervention products.

2. The computer method of claim 1, wherein said digital perceived risk hierarchy inventory further comprises data indicative of one or more perceptions of social and cultural environmental factors held by said human risk population member, and demographic data associated with said human risk population member.

3. The computer method of claim 2, further comprising the step of establishing at said processor of said predictive risk assessment unit a risk predictive quotient matrix assigning a numeric risk prediction quotient to each of one or more risk segments in said risk predictive quotient matrix.

4. The computer method of claim 3, wherein said risk predictive quotient matrix further comprises a statistical weight assigned by said processor of said predictive risk assessment unit to each said numeric risk prediction quotient for each said risk segment in said risk predictive quotient matrix.

5. The computer method of claim 4, wherein identifying one or more intervention products that are calculated to mitigate one or more negative or harmful behaviors associated with said perceived risk hierarchy inventory further comprises identifying one or more intervention products that are calculated to mitigate behaviors included in said risk segments of said risk predictive quotient matrix that have a numeric value that is higher than a predetermined value.

6. The computer method of claim 1, further comprising transmitting said individual risk portfolio to an intervention partner computer.

7. The computer method of claim 6, further comprising receiving at said predictive risk assessment unit from said intervention partner computer a numeric assessment of success in applying said one or more intervention products in said individual risk portfolio.

8. The computer method of claim 7, further comprising the step of in response to receiving said numeric assessment of success in applying said one or more intervention products in said individual risk portfolio, recalibrating at said predictive risk assessment unit statistical values applied by said predictive risk assessment unit when analyzing said digital perceived risk hierarchy inventory.

9. A computer system for predictive assessment and intervention, comprising:
a predictive risk assessment unit having a processor and a memory; and
a survey device in data communication with said predictive risk assessment unit;
wherein said processor of said predictive risk assessment unit includes computer instructions configured to:
receive from said survey device a digital perceived risk hierarchy inventory associated with a human risk population member;
analyze said digital perceived risk hierarchy inventory to identify one or more intervention products that are calculated to mitigate one or more negative or harmful behaviors associated with said perceived risk hierarchy inventory; and
store in said memory an individual risk portfolio associated with said human risk population member, said individual risk portfolio including said one or more intervention products.

10. The computer system of claim 9, wherein said digital perceived risk hierarchy inventory further comprises data indicative of one or more perceptions of social and cultural environmental factors held by said human risk population member, and demographic data associated with said human risk population member.

11. The computer method of claim 10, wherein said computer instructions are further configured to establish a risk predictive quotient matrix assigning a numeric risk prediction quotient to each of one or more risk segments in said risk predictive quotient matrix.

12. The computer method of claim 11, wherein said risk predictive quotient matrix further comprises a statistical weight assigned by said processor of said predictive risk assessment unit to each said numeric risk prediction quotient for each said risk segment in said risk predictive quotient matrix.

13. The computer method of claim 12, wherein identifying one or more intervention products that are calculated to mitigate one or more negative or harmful behaviors associated with said perceived risk hierarchy inventory further comprises identifying one or more intervention products that are calculated to mitigate behaviors included in said risk segments of said risk predictive quotient matrix that have a numeric value that is higher than a predetermined value.

14. The computer method of claim 9, wherein said computer instructions are further configured to transmit said individual risk portfolio to an intervention partner computer.

15. The computer method of claim 14, wherein said computer instructions are further configured to receive from said intervention partner computer a numeric assessment of success in applying said one or more intervention products in said individual risk portfolio.

16. The computer method of claim 15, wherein said computer instructions are further configured to, in response to receiving said numeric assessment of success in applying said one or more intervention products in said individual risk portfolio, recalibrate at said predictive risk assessment unit statistical values applied by said predictive risk assessment unit when analyzing said digital perceived risk hierarchy inventory.
